(19) United States
(12) Patent Application Publication
(10) Pub. No.: US 2024/0307135 A1
(43) Pub. Date: Sep. 19, 2024

(54) DELIVERY APPARATUS FOR ELONGATED MEDICAL INSTRUMENT

(71) Applicant: SHENZHEN INSTITUTE OF ADVANCED BIOMEDICAL ROBOT CO., LTD., SHENZHEN (CN)

(72) Inventors: LIANGZHENG YANG, SHENZHEN (CN); QUN LIU, SHENZHEN (CN); GANG YAO, SHENZHEN (CN); WEINAN YANG, SHENZHEN (CN)

(21) Appl. No.: 18/631,076

(22) Filed: Apr. 10, 2024

Related U.S. Application Data
(63) Continuation of application No. PCT/CN2023/095356, filed on May 19, 2023.

(30) Foreign Application Priority Data
Mar. 13, 2023 (CN) 202310282541.7

(51) Int. Cl. A61B 34/30

(52) U.S. Cl. CPC A61B 34/30; A61B 2034/301

(57) ABSTRACT

A delivery apparatus for elongated medical instruments includes a first driving mechanism, a second driving mechanism, and a third driving mechanism. A rotating and delivering assembly configured to clamp a medical instrument is not arranged on the second driving mechanism, whereas a third rotating and delivering assembly is arranged on the third driving mechanism. Under different intervention modes, the third rotating and delivering assembly may be configured to clamp, rotate and deliver a third medical instrument, or the third rotating and delivering assembly, upon manipulating the third medical instrument, clamp, rotates and delivers a fourth medical instrument. A third delivering assembly is configured to clamp the third medical instrument when the third rotating and delivering assembly manipulates the fourth medical instrument, such that the volume and weight of the entire slave-end apparatus are reduced and the costs are lowered.

### CROSS-REFERENCE OF RELATED APPLICATIONS

This application a continuation application of International Application No. PCT/CN2023/095356 filed on May 19, 2023, which claims priority to Chinese Patent Application No. 202310282541.7, filed on Mar. 13, 2023 and entitled “DELIVERY APPARATUS FOR ELONGATED MEDICAL INSTRUMENT,” the disclosures of which are incorporated herein by reference in their entireties.

### TECHNICAL FIELD

The present disclosure relates to the technical field of surgical robots, and in particular, relates to a delivery apparatus for elongated medical instruments.

### BACKGROUND

Interventional therapy is a minimally invasive treatment using modern high-tech means. Under the guidance of medical imaging equipment, special precision devices, such as catheters and guidewires, are introduced into the human body to diagnose and locally treat diseases in vivo.

During the operation of a surgical robot, it is necessary to deliver medical instruments to lesions under cooperation of the medical instruments (such as guidewires and catheters). With the continuous advancement of medical technologies and the needs of operation, the intervention mode of “one catheter and one guidewire” of vascular interventional surgery fails to meet the needs of doctors' operation. However, for intervention modes of “two catheters and one guidewire”, “three catheters and one guidewire” and the like “multi-catheter and one guidewire”, more choices are provided for doctors in treatment schemes in operation. Therefore, these intervention modes are popular with doctors. However, Chinese patent “CN202210326352.0” discloses a slave-end apparatus for an interventional surgical robot, wherein a first movement driving apparatus and a second movement driving apparatus are both provided with a driving unit for rotating and delivering a guidewire, resulting in a large volume, heavy weight, high costs of the whole slave-end apparatus and much waste of catheters. In addition, the slave-end apparatus in the related art fails to implement the intervention mode of “one catheter and two guidewires”.

### SUMMARY

A main object of the present disclosure is to provide a delivery apparatus for an elongated medical instrument, which can solve the technical problems in the related art that the slave-end apparatus is bulky, heavy and costly and catheters are wasted, and that the slave-end apparatus fails to implement the intervention mode of “one catheter and two guidewires”.

Embodiments of the present disclosure provide a delivery apparatus for elongated medical instruments, applicable to cooperative manipulation for a plurality of elongated medical instruments. The delivery apparatus includes:
a first driving mechanism, including a first delivering assembly, wherein the first delivering assembly is configured to clamp one end of a first medical instrument and deliver the first medical instrument;
a second driving mechanism, including a second rotating assembly and a second delivering assembly, wherein the second rotating assembly is configured to clamp the other end of the first medical instrument and rotate the first medical instrument, and the second delivering assembly is configured to clamp one end of a second medical instrument and deliver the second medical instrument; and
a third driving assembly, including a third rotating and delivering assembly, a third rotating assembly, and a third delivering assembly, wherein the third rotating assembly is configured to clamp the other end of the second medical instrument and rotate the second medical instrument, the third rotating and delivering assembly is configured to clamp a third medical instrument and rotate and deliver the third medical instrument, or the third rotating and delivering assembly is configured to clamp a fourth medical instrument and rotate and deliver the fourth medical instrument upon completion of manipulating the third medical instrument, and the third delivering assembly is configured to clamp the third medical instrument while the third rotating and delivering assembly is manipulating the fourth medical instrument;
wherein the plurality of elongated medical instruments are delivered and rotated by using the first driving mechanism, the second driving mechanism, and the third driving mechanism.

In some embodiments, in cooperative manipulation for the first medical instrument and the third medical instrument by using the first driving mechanism, the second driving mechanism, and the third driving mechanism, the second driving mechanism and the third driving mechanism remain stationary relative to each other, first the third rotating and delivering assembly clamps, rotates and delivers the third medical instrument such that a distance by which an end portion of the third medical instrument is exposed from an end portion of the first medical instrument is a predetermined distance, and then the first medical instrument and the third medical instrument are simultaneously delivered to a designated position.

In some embodiments, in cooperative manipulation of the first medical instrument, the third medical instrument, and the fourth medical instrument by using the first driving mechanism, the second driving mechanism, and the third driving mechanism, the second driving mechanism and the third driving mechanism remain stationary relative to each other, first the third rotating and delivering assembly clamps, rotates and delivers the third medical instrument such that a distance by which an end portion of the third medical instrument is exposed from an end portion of the first medical instrument is a predetermined distance, then the first medical instrument and the third medical instrument are simultaneously delivered to a first designated position, subsequently the third rotating and delivering assembly clamps, rotates and delivers the third medical instrument such that the third medical instrument is delivered to a second target position, and finally the third medical instrument is clamped on the third delivering assembly and the third rotating and delivering assembly clamps, rotates and delivers the fourth medical instrument.

In some embodiments, the second driving mechanism further includes a rapid exchange delivering assembly, configured to clamp and deliver a rapid exchange catheter.

In some embodiments, in cooperative manipulation for the first medical instrument, the second medical instrument, and the third medical instrument by using the first driving mechanism, the second driving mechanism, and the third driving mechanism, the second driving mechanism and the third driving mechanism independently move relative to each other, first the third rotating and delivering assembly clamps, rotates and delivers the third medical instrument such that a distance by which an end portion of the third medical instrument is exposed from an end portion of the first medical instrument is a first predetermined distance, then the second delivering assembly and the third rotating assembly cooperatively deliver the second medical instrument such that a distance by which the second medical instrument is exposed from the first medical instrument is a second predetermined distance, subsequently the third rotating and delivering assembly clamps, rotates and delivers the third medical instrument such that a distance by which an end portion of the third medical instrument is exposed from an end portion of the second medical instrument is a third predetermined distance, and finally the first medical instrument, the second medical instrument, and the third medical instrument are simultaneously delivered to designated positions.

In some embodiments, the third delivering assembly is configured to clamp and deliver a rapid exchange catheter.

In some embodiments, the third rotating and delivering assembly includes a rotating unit and a delivering unit; wherein the rotating unit and the delivering unit are assembled to an integral structure or are independently arranged.

In some embodiments, the delivery apparatus further includes: a front-end securing mechanism; wherein the front-end securing mechanism is arranged on a side, away from the second driving mechanism, of the first driving mechanism.

In some embodiments, in cooperative manipulation for the first medical instrument, the second medical instrument, the third medical instrument, and a fifth medical instrument by using the first driving mechanism, the second driving mechanism, and the third driving mechanism, the first driving mechanism delivers the first medical instrument to a designated position, and the first medical instrument is clamped on the front-end securing mechanism.

In some embodiments, the delivery apparatus further includes: a sheath mounting base including a mounting portion and a connecting portion; wherein the mounting portion is configured to mount a puncture sheath, the mounting portion is connected to the front-end securing mechanism via the connecting portion, and during delivery, the first driving mechanism drives the first medical instrument to extend through the puncture sheath.

Compared to the related art, the present disclosure provides a delivery apparatus for elongated medical instruments. The delivery apparatus includes a first driving mechanism, a second driving mechanism, and a third driving mechanism. A rotating and delivering assembly configured to clamp a medical instrument is not arranged on the second driving mechanism, whereas a third rotating and delivering assembly is arranged on the third driving mechanism. Under different intervention modes, the third rotating and delivering assembly may be configured to clamp, rotate and deliver a third medical instrument, or the third rotating and delivering assembly, upon manipulating the third medical instrument, clamp, rotates and delivers a fourth medical instrument. A third delivering assembly is configured to clamp the third medical instrument when the third rotating and delivering assembly manipulates the fourth medical instrument, such that the length of the second driving mechanism is decreased, and the number of motors and waste in terms of length of the second driving mechanism are both reduced. In this way, the volume and weight of the entire slave-end apparatus are reduced, and the costs are lowered. In addition, an intervention mode of “one catheter and two guidewires” are implemented on the delivery apparatus.

### BRIEF DESCRIPTION OF THE DRAWINGS

FIG. 1 is a schematic structural diagram illustrating delivering “one catheter and one guidewire” by a delivery apparatus for elongated medical instruments according to some embodiments of the present disclosure;

FIG. 2 is a schematic structural diagram illustrating delivering “one catheter and two guidewires” by a delivery apparatus for elongated medical instruments according to some embodiments of the present disclosure;

FIG. 3 is a schematic structural diagram illustrating delivering “two catheters and one guidewire” by a delivery apparatus for elongated medical instruments according to some embodiments of the present disclosure;

FIG. 4 is a partial enlarged view of part A in FIG. 3;

FIG. 5 is a partial enlarged view of part B in FIG. 3; and

FIG. 6 is a partial enlarged view of part C in FIG. 3.

Reference numerals and denotations thereof. 1—first driving assembly; 11—first delivering assembly; 2—second driving mechanism; 21—second rotating assembly; 211—T-valve; 22—second delivering assembly; 23—rapid exchange delivering assembly; 3—third driving mechanism; 31—third rotating and delivering assembly; 311—rotating unit; 312—delivering unit; 32—third rotating assembly; 33—third delivering assembly; 4—frame; 5—sheath mounting base; 51—mounting portion; 52—connecting portion; 6—front—end securing mechanism; 101—first medical instrument; 102—second medical instrument; 103—third medical instrument; 104—fourth medical instrument; 106—rapid exchange catheter.

The attainment of the objectives, functional features and advantages of the present disclosure are further described hereinafter with reference to the specific embodiments and the accompanying drawings.

### DETAILED DESCRIPTION

It should be understood that the specific embodiments described herein are only intended to explain the present disclosure instead of limiting the present disclosure.

The technical solutions contained in the embodiments of the present disclosure are described in detail clearly and completely hereinafter with reference to the accompanying drawings for the embodiments of the present disclosure. Apparently, the described embodiments are only a portion of embodiments of the present disclosure, but not all the embodiments of the present disclosure. Based on the embodiments of the present disclosure, all other embodiments derived by persons of ordinary skill in the art without any creative efforts shall fall within the protection scope of the present disclosure.

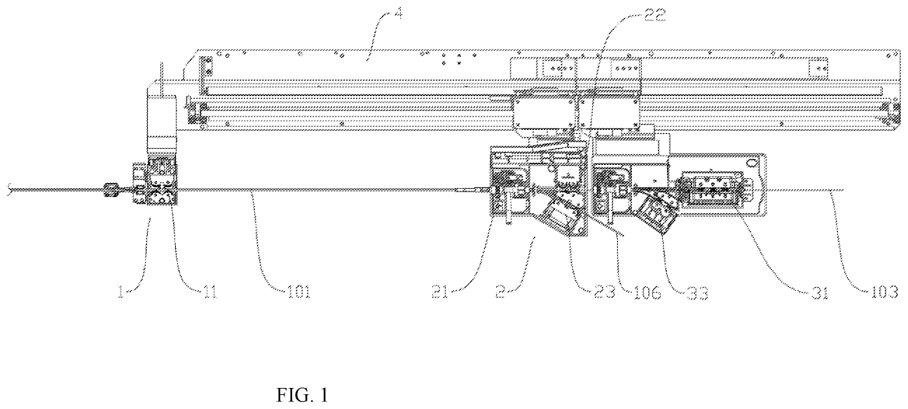

Referring to FIG. 1 to FIG. 3, some embodiments of the present disclosure provide a delivery apparatus for elongated medical instruments, applicable to cooperative manipulation for a plurality of elongated medical instruments. The delivery apparatus includes:
a first driving mechanism 1, including a first delivering assembly 11, wherein the first delivering assembly 11 is configured to clamp one end of a first medical instrument 101 and deliver the first medical instrument 101;
a second driving mechanism 2, including a second rotating assembly 21 and a second delivering assembly 22, wherein the second rotating assembly 21 is configured to clamp the other end of the first medical instrument 101 and rotate the first medical instrument 101, and the second delivering assembly 22 is configured to clamp one end of a second medical instrument 102 and deliver the second medical instrument 102; and
a third driving assembly 3, including a third rotating and delivering assembly 31, a third rotating assembly 32, and a third delivering assembly 33, wherein the third rotating assembly 32 is configured to clamp the other end of the second medical instrument 102 and rotate the second medical instrument 102, the third rotating and delivering assembly 31 is configured to clamp a third medical instrument 103 and rotate and deliver the third medical instrument 103, or the third rotating and delivering assembly 31 is configured to clamp a fourth medical instrument 104 and rotate and deliver the fourth medical instrument 104 upon completion of manipulating the third medical instrument 103, and the third delivering assembly 33 is configured to clamp the third medical instrument 103 while the third rotating and delivering assembly 31 is manipulating the fourth medical instrument 104;
wherein the plurality of elongated medical instruments are delivered and rotated by using the first driving mechanism 1, the second driving mechanism 2, and the third driving mechanism 3.

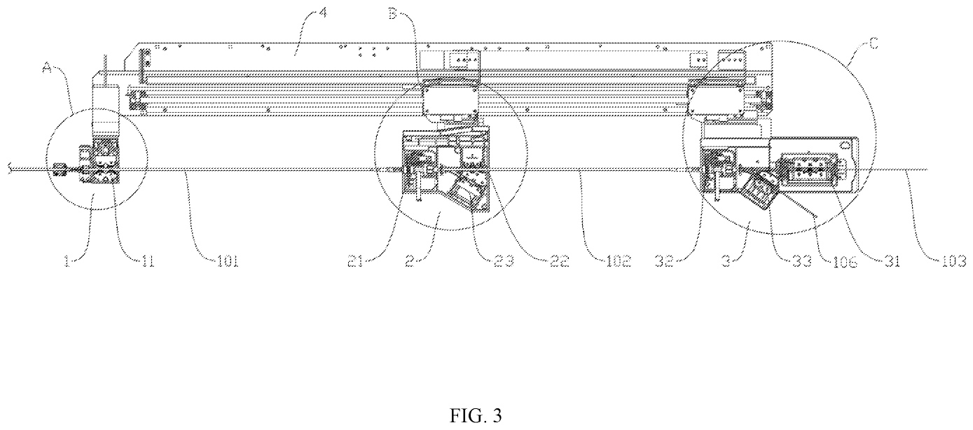

Referring to FIG. 3, in some exemplary embodiments, the delivery apparatus includes a frame 4. The first driving mechanism 1 is secured onto the frame 4, and clamps one end of the first medical instrument 101 and delivers the first medical instrument 101. The second driving mechanism 2 is movably arranged on the frame 4, and clamps the other end of the first medical instrument 101 and delivers the first medical instrument 101 by virtue of displacement on the frame 4. It should be noted herein that the first driving mechanism 1 is secured onto the frame 4, and delivers one end of the first medical instrument 101 via an internal structure thereof; however, different from the way of delivering the other end of the first medical instrument 101 by the first driving mechanism 1, the second driving mechanism 2 delivers the other end of the first medical instrument 101 by virtue of displacement on the frame 4. Specifically, the internal structure includes a first delivering assembly 11 configured to deliver the first medical instrument 101. The first delivering assembly 11 includes a first roller set. The one end of the first medical instrument 101 is delivered by virtue of a friction between the first roller set and the first medical instrument 101. The second driving mechanism 2 is driven to displace by a driving device (for example, a linear module, a lead screw motor, or the like) arranged on the frame 4, and the other end of the first medical instrument 101 is delivered by virtue of an overall displacement of the second driving mechanism 2 on the frame 4. The first driving mechanism 1 needs to deliver the catheter of the first medical instrument 101 at a speed consistent with a speed at which the second driving mechanism 2 delivers the first medical instrument 101, such that the first medical instrument 101 between the first driving mechanism 1 and the second driving mechanism 2 constantly remains straight. In addition, likewise, the second driving mechanism 2 is also capable of clamping one end of the second medical instrument 102, and the third driving mechanism 3 is movably arranged on the frame 4, and clamps the other end of the elongated second medical instrument 102 and delivers the second medical instrument 12 by virtue of displacement on the frame 4. The principles for delivering the second medical instrument 102 by the second driving mechanism 2 and the third driving mechanism 3 are approximately the same as the principles for delivering the first medical instrument 101 by the first driving mechanism 1 and the second driving mechanism 2. An internal structure of the second driving mechanism 2 includes a second delivering assembly 22 configured to deliver the second medical instrument 102 and a second rotating assembly 21 configured to rotate the first medical instrument 101. The second delivering assembly 22 includes a second roller set. One end of the second medical instrument 102 is delivered by virtue of a friction between the second roller set and the second medical instrument 102. The third driving mechanism 3 is driven to displace by a driving device (for example, a linear module, a lead screw motor, or the like) arranged on the frame 4, and the other end of the second medical instrument 102 is delivered by virtue of an overall displacement of the third driving mechanism 3 on the frame 4. In addition, the second rotating assembly 21 includes a rotating unit. The rotating unit is configured to drive a T valve 211 to rotate, and the first medical instrument 101 secured to the T valve 211 is rotated under driving of the T valve. In this way, upon being pushed forward by a specific distance, the first medical instrument 101, reaching a bent portion of a blood vessel or a branch of the blood vessel, needs to be rotated to continue its travel along the bent portion of the blood vessel or enter a target branch. The third rotating assembly 32 of the third driving mechanism 3 also includes a rotating unit, configured to rotate the second medical instrument 102. The third delivering assembly 33 includes a third roller set, and the third medical instrument 103 may be secured by a clamping force between rollers of the third roller set, such that the subsequent operations are eased.

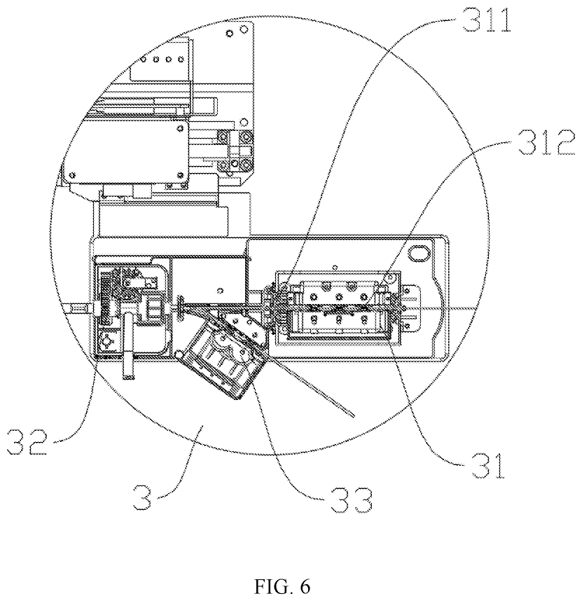

Referring to FIG. 6, the third rotating and delivering assembly 31 has a complicated structure. Specifically, the third rotating and delivering assembly 31 includes a rotating unit 311 and a delivering unit 312. The rotating unit 311 and the delivering unit 312 are assembly to an integral structure, or the rotating unit 311 and the delivering unit 312 are separately arranged. When being pushed forward by a specific distance and reaching the bent portion of the blood vessel or the branch of the blood vessel, the third medical instrument 103 needs to be rotated to change its direction of the head thereof, such that the third medical instrument 103 needs to continue its travel along the bent portion of the blood vessel or enter the target branch. The third medical instrument 103 is rotated by using the rotating unit 311, and the third medical instrument 103 is delivered by using the delivering unit 312. Since the third driving mechanism 3 is additionally provided with the third rotating and delivering assembly 31 compared to the second driving mechanism 2, the volume and weight of the third driving mechanism 3 are increased over the second driving mechanism 2. In the related art, for intervention of a plurality of medical instruments, a rotating and delivering assembly having the similar function of the third rotating and delivering assembly 31 is also mounted on the second driving mechanism 2, such that the volume and weight of the second driving mechanism 2 are also increased, and hence the manufacture costs are increased accordingly. In addition, since the volume of the second driving mechanism 2 is over-large, a lot of medical instruments are not reasonably utilized, and thus waste is caused. Therefore, according to the present disclosure, the rotating and delivering assembly is only arranged on the third driving mechanism 3, such that the length of the second driving mechanism 2 is decreased, and the number of motors and waste in terms of length of the second driving mechanism 2 are both reduced. In this way, the volume and weight of the entire slave-end apparatus are reduced, and the costs are lowered. In addition, by cooperation between the second driving mechanism 2 and the third driving mechanism 3, an intervention mode of “one catheter and two guidewires” are implemented on the delivery apparatus.

Specifically, according to the present disclosure, the first medical instrument 101 is a first catheter, the second medical instrument 102 is a second catheter, the third medical instrument 103 is a first guidewire, and the fourth medical instrument 104 is a second guidewire.

Referring to FIG. 1, in some exemplary embodiments, in cooperative manipulation for the first medical instrument 101 and the third medical instrument 103 by using the first driving mechanism 1, the second driving mechanism 2, and the third driving mechanism 3, the second driving mechanism 2 and the third driving mechanism 3 remain stationary relative to each other, first the third rotating and delivering assembly 31 clamps, rotates and delivers the third medical instrument 103 such that a distance by which an end portion of the third medical instrument 103 is exposed from an end portion of the first medical instrument 101 is a predetermined distance, and then the first medical instrument 101 and the third medical instrument 103 are simultaneously delivered to a designated position. In specific application scenarios, an intervention mode of “one catheter and one guidewire” are typically used for a preoperative contrast imaging examination. In this examination, the third driving mechanism 3 is attached to the second driving mechanism 2 and combined with the second driving mechanism 2 to form an integral structure (in this case, the third driving mechanism 3 may be considered as a portion of the second driving mechanism 2), and the second driving mechanism 2 and the third driving mechanism 3 remains stationary relative to each other. That is, the second driving mechanism 2 and the third driving mechanism 3 move synchronously. First, the third rotating and delivering assembly 31 clamps, rotates and delivers the first guidewire, and when the first guidewire is exposed from an end portion of the first catheter by a distance of 100 to 200 mm, one end of the first catheter is delivered by using the first delivering assembly 11 and the other end of the first catheter is delivered by the second rotating assembly 21 which is driven by the entire movement of the second driving mechanism 2. In this process, a delivering speed of the first delivering assembly 11 is equal to an overall movement speed of the second driving mechanism 2. The second driving mechanism 2 in movement drives the third driving mechanism 3 to move synchronously. Therefore, the guidewire clamped on the third driving mechanism 3 is delivered synchronously with the first catheter. When the first catheter and the first guidewire are delivered together to the aortic arch, the first guidewire is withdrawn by using the third rotating and delivering assembly 31, and then a contrast agent is injected through the first catheter for contrast imaging. Upon completion of the contrast imaging, the first guidewire is delivered to be exposed from the end portion of the first catheter by a distance of 100 to 200 mm by using the third rotating and delivering assembly 31, and then the first catheter and the first guidewire are together withdrawn from the human body by using the first delivering assembly 11 and the second driving mechanism 2. In the above process, since the second driving mechanism 2 and the third driving mechanism 3 remain stationary relative to each other, when the second driving mechanism 2 delivers the first catheter, the first guidewire clamped on the third driving mechanism 3 is delivered synchronously with the first catheter. With the blood vessel as a reference, where only the first catheter needs to be delivered in the blood vessel whereas the first guidewire remains stationary relative to the blood vessel, the first guidewire needs to be reversely delivered by using the third rotating and delivering assembly 31, such that the first guidewire remains stationary relative to the blood vessel. Likewise, in other intervention modes, where the first guidewire is driven due to structural linkage but is expected to remain stationary relative to the blood vessel, the first guidewire may also be reversely delivered by using the third rotating and delivering assembly 31, such that the first guidewire remains stationary relative to the blood vessel. In addition, in the embodiments, the second driving mechanism 2 further includes a rapid exchange delivering assembly 23, wherein the rapid exchange delivering assembly 23 is configured to clamp and deliver a rapid exchange catheter 106. The rapid exchange delivering assembly 23 includes a roller set, and a tail end of the rapid exchange catheter 106 is delivered by virtue of a friction effect between the roller set and the rapid exchange catheter 106. In the intervention mode of “one catheter and one guidewire”, deliver of the rapid exchange catheter 106 is completed on the second driving mechanism 2. Specifically, where a rapid exchange operation is needed, a tail end of the first guidewire is led in through a head end of the rapid exchange catheter 106 and then led out from a side hole of the rapid exchange catheter 106, and finally the tail end of the rapid exchange catheter 106 is mounted on the rapid exchange delivering assembly 23. By manipulating the rapid exchange delivering assembly 23, the rapid exchange catheter 106 is delivered along the first guidewire.

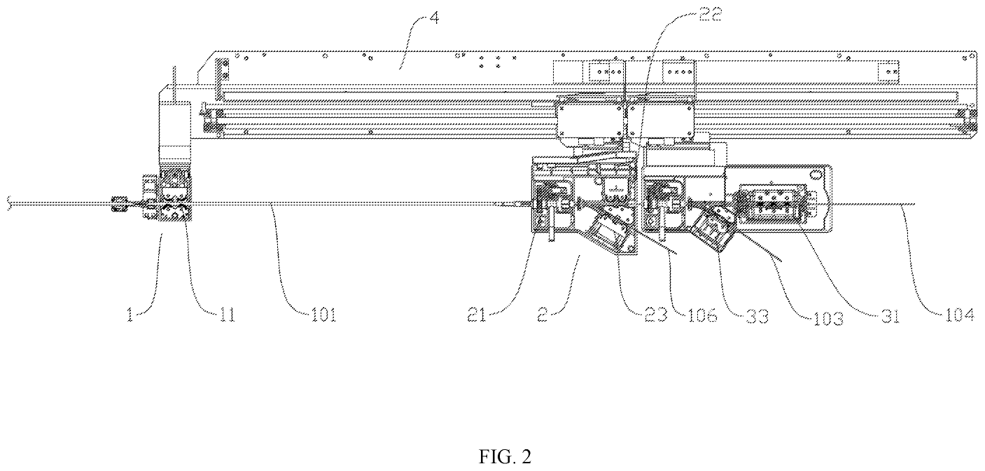

Referring to FIG. 2, in some exemplary embodiments, in cooperative manipulation for the first medical instrument 101, the third medical instrument 103, and the fourth medical instrument 104 by using the first driving mechanism 1, the second driving mechanism 2, and the third driving mechanism 3, the second driving mechanism 2 and the third driving mechanism 3 remain stationary relative to each other, first the third rotating and delivering assembly 31 clamps, rotates and delivers the third medical instrument 103 such that a distance by which an end portion of the third medical instrument 103 is exposed from an end portion of the first medical instrument 101 is a predetermined distance, then the first medical instrument 101 and the third medical instrument 103 are simultaneously delivered to a first designated position, subsequently the third rotating and delivering assembly 31 clamps, rotates and delivers the third medical instrument 103 such that the third medical instrument 103 is delivered to a second target position, and finally the third medical instrument 103 is clamped on the third delivering assembly 33 and the third rotating and delivering assembly 31 clamps, rotates and delivers the fourth medical instrument 104. In specific application scenarios, an intervention mode of “one catheter and one guidewire” are typically used for treatment of coronary bifurcation lesions. In this examination, the third driving mechanism 3 is attached to the second driving mechanism 2 and combined with the second driving mechanism 2 to form an integral structure (in this case, the third driving mechanism 3 may be considered as a portion of the second driving mechanism 2), and the second driving mechanism 2 and the third driving mechanism 3 remains stationary relative to each other. That is, the second driving mechanism 2 and the third driving mechanism 3 move synchronously. First, the third rotating and delivering assembly 31 clamps, rotates and delivers the first guidewire, and when the first guidewire is exposed from the end portion of the first catheter by a distance of 100 to 200 mm, the first catheter and the first guidewire are together delivered to the aortic arch. This process is the same as the specific process of delivering the first catheter and the first guidewire in the above embodiments, which is not described herein any further. Then, the first guidewire is controlled by using the third rotating and delivering assembly 31 and the first catheter is controlled by using the first delivering assembly 11 and the second rotating assembly 21 to form cooperation, and the first guidewire and the first catheter are delivered to a coronary bifurcation lesion. When the first guidewire enters a coronary bifurcation lesion A in the blood vessel, the first guidewire is removed from the third rotating and delivering assembly 31 and clamped on the third delivering assembly 33, and then the second guidewire is delivered by using the third rotating and delivering assembly 31 to the blood vessel with a coronary bifurcation lesion B in the blood vessel. In the embodiments, the second driving mechanism 2 further includes a rapid exchange delivering assembly 23, wherein the rapid exchange delivering assembly 23 is configured to clamp and deliver a rapid exchange catheter 106. Specifically, the rapid exchange delivering assembly 23 is arranged on a side of the second delivering assembly 22. The rapid exchange delivering assembly 23 includes a roller set, and the rapid exchange catheter 106 is delivered by virtue of a friction effect between the roller set and the rapid exchange catheter 106. In the treatment process, where a rapid exchange operation is needed, a tail end of the second guidewire is led in through the head end of the rapid exchange catheter 106 and then led out from the side hole of the rapid exchange catheter 106, and the rapid exchange catheter 106 clamped on the rapid exchange delivering assembly 23 treats the coronary bifurcation lesion B using the second guidewire. Where the blood vessel A is found to be narrow during the treatment, the tail end of the first guidewire is led in through the head end of the rapid exchange catheter 106 and then led out from the side hole of the rapid exchange catheter 106, and the rapid exchange catheter 106 clamped on the rapid exchange delivering assembly 23 treats the coronary bifurcation lesion A using the first guidewire. Upon completion of the treatment, the rapid exchange catheter 106 is withdrawn, and then the second guidewire, the first catheter, and the first guidewire are all withdrawn. In this process, the medical instruments are withdrawn by the corresponding driving assemblies, which are not described herein any further.

Referring to FIG. 3, in some exemplary embodiments, in cooperative manipulation for the first medical instrument 101, the second medical instrument 102, and the third medical instrument 103 by using the first driving mechanism 1, the second driving mechanism 2, and the third driving mechanism 3, the second driving mechanism 2 and the third driving mechanism 3 independently move relative to each other, first the third rotating and delivering assembly 31 clamps, rotates and delivers the third medical instrument 103 such that a distance by which an end portion of the third medical instrument 103 is exposed from an end portion of the first medical instrument 101 is a first predetermined distance, then the second delivering assembly 22 and the third rotating assembly 32 cooperatively deliver the second medical instrument 102 such that a distance by which the second medical instrument 102 is exposed from the first medical instrument 101 is a second predetermined distance, subsequently the third rotating and delivering assembly 31 clamps, rotates and delivers the third medical instrument 103 such that a distance by which an end portion of the third medical instrument 103 is exposed from an end portion of the second medical instrument 102 is a third predetermined distance, and finally the first medical instrument 101, the second medical instrument 102, and the third medical instrument 103 are simultaneously delivered to designated positions. In specific application scenarios, mechanical thrombectomy is for intracranial vascular stenosis lesions, and the intervention mode of “two catheters and one guidewire” generally needs to be used. In this process, the second driving mechanism 2 and the third driving mechanism 3 independently moves relative to each other. First, the third rotating and delivering assembly 31 clamps, rotates and delivers the first guidewire, and when the first guidewire is exposed from an end portion of the first catheter by a distance of 100 to 200 mm, one end of the second catheter is delivered by using the second delivering assembly 22 and the other end of the second catheter is delivered by the second rotating assembly 21 which is driven by the entire movement of the third driving mechanism 3. In this process, a delivering speed of the second delivering assembly 22 is equal to an overall movement speed of the third driving mechanism 3. When the second catheter is exposed from the end portion of the first catheter by a distance of 100 mm and the first guidewire delivered by the third rotating and delivering assembly 31 to be exposed from the end portion of the second catheter by a distance of 200 mm, by cooperation of the first driving mechanism 1, the second driving mechanism 2, and the third driving mechanism 3, the first catheter, the second catheter, and the first guidewire are simultaneously delivered to a stable position of the left carotid artery (or stable position of the right carotid artery, which depends on the position of the lesion). Then, the second catheter and the first guidewire are withdrawn, and other instruments are delivered for the thrombectomy procedure. In the embodiments, where a rapid exchange operation is needed, the third delivering assembly 33 is configured to clamp and deliver the rapid exchange catheter 106. The third delivering assembly 33 includes a roller set, and the rapid exchange catheter 106 is delivered by virtue of a friction effect between the roller set and the rapid exchange catheter 106.

In addition, the delivery apparatus further includes a front-end securing mechanism 6. The front-end securing mechanism 6 is arranged on a side, away from the second driving mechanism 2, of the first driving mechanism 1.

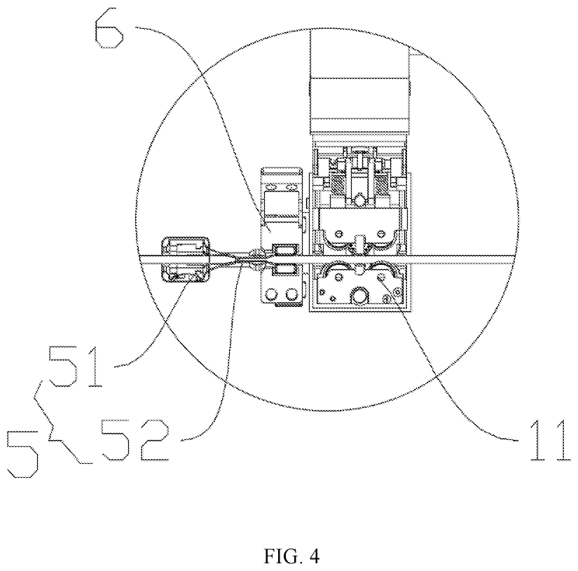

Referring to FIG. 3 to FIG. 5, in some exemplary embodiments of the present disclosure, by cooperative manipulation for the first medical instrument 101, the second medical instrument 102, the third medical instrument 103, and a fifth medical instrument (not illustrated in the drawings) by using the first driving mechanism 1, the second driving mechanism 2, and the third driving mechanism 3, the first driving mechanism 1 delivers the first medical instrument 101 to a designated position, and the first medical instrument 101 is clamped on the front-end securing mechanism 6. In the embodiments, the fifth medical instrument is a third catheter (not illustrated in the drawings), the T valve 211 is secured to one end of the first catheter, and during manipulation, the second rotating assembly rotates the T valve 211 to drive the first catheter to rotate. In specific application scenarios, where an intervention mode of “three catheters and one guidewire” is needed, a front-end securing mechanism 6 may be arranged to secure the T valve 211, such that the first catheter is secured. In specific application scenarios, mechanical thrombectomy for acute stroke is for intracranial vascular stenosis lesions, and the intervention mode of “three catheters and one guidewire” generally needs to be used. In this process, the second driving mechanism 2 and the third driving mechanism 3 independently moves relative to each other. First, in the intervention mode of “two catheters and one guidewire”, the first guidewire, the first catheter, and the second catheter are delivered. The third rotating and delivering assembly 31 clamps, rotates and delivers the first guidewire, and when the first guidewire is exposed from an end portion of the first catheter by a distance of 100 to 200 mm, one end of the second catheter is delivered by using the second delivering assembly 22 and the other end of the second catheter is delivered by the third rotating assembly 32 which is driven by the entire movement of the third driving mechanism 3. In this process, a delivering speed of the second delivering assembly 22 is equal to an overall movement speed of the third driving mechanism 3. When the second catheter is exposed from the end portion of the first catheter by a distance of 100 mm and the first guidewire delivered by the third rotating and delivering assembly 31 to be exposed from the end portion of the second catheter by a distance of 200 mm, by cooperation of the first driving mechanism 1, the second driving mechanism 2, and the third driving mechanism 3, the first catheter, the second catheter, and the first guidewire are simultaneously delivered to a stable position of the left carotid artery (or stable position of the right carotid artery, which depends on the position of the lesion). Subsequently, the second catheter and the first guidewire are withdrawn, the first catheter is removed from the first delivering assembly 11 and the second rotating assembly 21, wherein one end of the first catheter is connected to the T valve 211, and then the T valve 211 is secured to the front-end securing mechanism 6. Afterwards, the fourth catheter (correspondingly mounted at a position where the first catheter is originally mounted), the fifth catheter (correspondingly mounted at a position where the second catheter is originally mounted), and the second guidewire (correspondingly mounted t a position where the first guidewire is originally mounted) are mounted. Likewise, in this process, the second driving mechanism 2 and the third driving mechanism 3 independently moves relative to each other. Still in the intervention mode of “two catheters and one guidewire”, the second guidewire, the fourth catheter, and the fifth catheter are delivered. First, the third rotating and delivering assembly 31 clamps, rotates and delivers the second guidewire, and when the second guidewire is exposed from an end portion of the fourth catheter by a distance of 100 to 200 mm, one end of the fifth catheter is delivered by using the second delivering assembly 22 and the other end of the fifth catheter is delivered by the second rotating assembly 32 which is driven by the entire movement of the third driving mechanism 3. In this process, a delivering speed of the second delivering assembly 22 is equal to an overall movement speed of the third driving mechanism 3. When the fifth catheter is exposed from the end portion of the fourth catheter by a distance of 100 mm and the second guidewire delivered by the third rotating and delivering assembly 31 to be exposed from the end portion of the fifth catheter by a distance of 200 mm, by cooperation of the first driving mechanism 1, the second driving mechanism 2, and the third driving mechanism 3, the fourth catheter, the fifth catheter, and the second guidewire are simultaneously delivered to a distal end of an intracranial lesion, and then the second guidewire is withdrawn and a thrombectomy stent is mounted for thrombectomy.

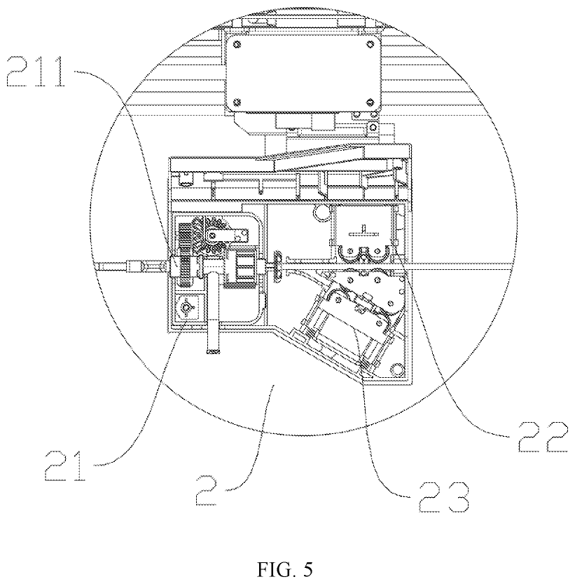

Referring to FIG. 5, in some exemplary embodiments, the delivery apparatus further includes a sheath mounting base 5. The sheath mounting base 5 includes a mounting portion 51 and a connecting portion 52. The mounting portion 51 is configured to mount a puncture sheath (not illustrated in the drawings). The mounting portion 51 is connected to the front-end securing mechanism 6 via the connecting portion 52, and during delivery, the first driving mechanism 1 drives the first medical instrument 101 to extend through the puncture sheath. In the related art, the puncture sheath is only secured to the wound of the human body using a medical adhesive tape, and is not secured to the slave-end apparatus. Therefore, where the slave-end apparatus moves, it is possible to pull a catheter sheath of the puncture sheath out of the blood vessel or cause the catheter sheath to a displacement. The mounting portion 51 is connected to the front-end securing mechanism 6 via the connecting portion 52, and the connecting portion 52 exerts a certain limiting and supporting effect on the catheter and the guidewire, which effectively solves the problem that in the related art that the puncture sheath is only bonded to the wound of the human body but is not secured to the slave end device, and hence the catheter sheath may be pulled to detach from the blood vessel or may be subject to a displacement when the slave-end apparatus moves.

Therefore, in the present disclosure, the front-end securing mechanism 6 implements different functions according to requirements in the process of operation. In practical applications, function switching is simple, the compatibility is strong, and the solution is applicable to different intervention modes of the medical instruments and mounting corresponding medical instruments, such that the operation efficiency is greatly improved.

It should be noted that, in this specification, terms “comprises,” “include,” “contain,” and any other variations thereof are intended to cover a non-exclusive inclusion, such that a process, method, article, or apparatus, that comprises, has, includes, or contains a list of elements does not include only those elements but may include other elements not expressly listed or inherent to such process, method, article, or apparatus. An element proceeded by “comprises . . . a,” “has . . . a,” “includes . . . a,” or “contains . . . a” does not, without more constraints, preclude the existence of additional identical elements in the process, method, article, or apparatus.

Described above are exemplary embodiments of the present disclosure, but are not intended to limit the scope of the present disclosure. Any equivalent structure or equivalent process variation made based on the specification and drawings of the present disclosure, which is directly or indirectly applied in other related technical fields, fall within the scope of the present disclosure.

1. A delivery apparatus for elongated medical instruments, applicable to cooperative manipulation for a plurality of elongated medical instruments, the apparatus comprising:
a first driving mechanism, comprising a first delivering assembly, wherein the first delivering assembly is configured to clamp one end of a first medical instrument and deliver the first medical instrument;
a second driving mechanism, comprising a second rotating assembly and a second delivering assembly, wherein the second rotating assembly is configured to clamp the other end of the first medical instrument and rotate the first medical instrument, and the second delivering assembly is configured to clamp one end of a second medical instrument and deliver the second medical instrument; and
a third driving assembly, comprising a third rotating and delivering assembly, a third rotating assembly, and a third delivering assembly, wherein the third rotating assembly is configured to clamp the other end of the second medical instrument and rotate the second medical instrument, the third rotating and delivering assembly is configured to clamp a third medical instrument and rotate and deliver the third medical instrument, or the third rotating and delivering assembly is configured to clamp a fourth medical instrument and rotate and deliver the fourth medical instrument upon completion of manipulating the third medical instrument, and the third delivering assembly is configured to clamp the third medical instrument while the third rotating and delivering assembly is manipulating the fourth medical instrument;
wherein the plurality of elongated medical instruments are delivered and rotated by using the first driving mechanism, the second driving mechanism, and the third driving mechanism.

2. The delivery apparatus for elongated medical instruments according to claim 1, wherein in cooperative manipulation for the first medical instrument and the third medical instrument by using the first driving mechanism, the second driving mechanism, and the third driving mechanism, the second driving mechanism and the third driving mechanism remain stationary relative to each other, first the third rotating and delivering assembly clamps, rotates and delivers the third medical instrument such that a distance by which an end portion of the third medical instrument is exposed from an end portion of the first medical instrument is a predetermined distance, and then the first medical instrument and the third medical instrument are simultaneously delivered to a designated position.

3. The delivery apparatus for elongated medical instruments according to claim 1, wherein in cooperative manipulation for the first medical instrument, the third medical instrument, and the fourth medical instrument by using the first driving mechanism, the second driving mechanism, and the third driving mechanism, the second driving mechanism and the third driving mechanism remain stationary relative to each other, first the third rotating and delivering assembly clamps, rotates and delivers the third medical instrument such that a distance by which an end portion of the third medical instrument is exposed from an end portion of the first medical instrument is a predetermined distance, then the first medical instrument and the third medical instrument are simultaneously delivered to a first designated position, subsequently the third rotating and delivering assembly clamps, rotates and delivers the third medical instrument such that the third medical instrument is delivered to a second target position, and finally the third medical instrument is clamped on the third delivering assembly and the third rotating and delivering assembly clamps, rotates and delivers the fourth medical instrument.

4. The delivery apparatus for elongated medical instruments according to claim 2, wherein when the second driving mechanism and the third driving mechanism remain stationary, the third driving mechanism is close to the second driving mechanism, and is integrated with the second driving mechanism.

5. The delivery apparatus for elongated medical instruments according to claim 2, wherein the second driving mechanism further comprises a rapid exchange delivering assembly, configured to clamp and deliver a rapid exchange catheter.

6. The delivery apparatus for elongated medical instruments according to claim 1, wherein in cooperative manipulation for the first medical instrument, the second medical instrument, and the third medical instrument by using the first driving mechanism, the second driving mechanism, and the third driving mechanism, the second driving mechanism and the third driving mechanism independently move relative to each other, first the third rotating and delivering assembly clamps, rotates and delivers the third medical instrument such that a distance by which an end portion of the third medical instrument is exposed from an end portion of the first medical instrument is a first predetermined distance, then the second delivering assembly and the third rotating assembly cooperatively deliver the second medical instrument such that a distance by which the second medical instrument is exposed from the first medical instrument is a second predetermined distance, subsequently the third rotating and delivering assembly clamps, rotates and delivers the third medical instrument such that a distance by which an end portion of the third medical instrument is exposed from an end portion of the second medical instrument is a third predetermined distance, and finally the first medical instrument, the second medical instrument, and the third medical instrument are simultaneously delivered to designated positions.

7. The delivery apparatus for elongated medical instruments according to claim 6, wherein the third delivering assembly is configured to clamp and deliver a rapid exchange catheter.

8. The delivery apparatus for elongated medical instruments according to claim 1, wherein the third rotating and delivering assembly comprises a rotating unit and a delivering unit; wherein the rotating unit and the delivering unit are assembled to an integral structure or are independently arranged.

9. The delivery apparatus for elongated medical instruments according to claim 1, further comprising: a front-end securing mechanism; wherein the front-end securing mechanism is arranged on a side, away from the second driving mechanism, of the first driving mechanism.

10. The delivery apparatus for elongated medical instruments according to claim 9, wherein in cooperative manipulation for the first medical instrument, the second medical instrument, the third medical instrument, and a fifth medical instrument by using the first driving mechanism, the second driving mechanism, and the third driving mechanism, the first driving mechanism delivers the first medical instrument to a designated position, and the first medical instrument is clamped on the front-end securing mechanism.

11. The delivery apparatus for elongated medical instruments according to claim 9, further comprising: a sheath mounting base comprising a mounting portion and a connecting portion; wherein the mounting portion is configured to mount a puncture sheath, the mounting portion is connected to the front-end securing mechanism via the connecting portion, and during delivery, the first driving mechanism drives the first medical instrument to extend through the puncture sheath.

12. The delivery apparatus for elongated medical instruments according to claim 1, comprises: a frame; wherein the first driving mechanism is secured onto the frame, and clamps one end of the first medical instrument and delivers the first medical instrument; and the second driving mechanism is movably arranged on the frame, and clamps the other end of the first medical instrument and delivers the first medical instrument by virtue of displacement on the frame.

13. The delivery apparatus for elongated medical instruments according to claim 12, wherein the first driving mechanism delivers one end of the first medical instrument by an internal structure thereof, the internal structure comprising a first delivering assembly configured to deliver the first medical instrument; wherein the first delivering assembly comprises a first roller set, and the one end of the first medical instrument is delivered by virtue of a friction between the first roller set and the first medical instrument; and the second driving mechanism is driven to displace by a driving device arranged on the frame, and the other end of the first medical instrument is delivered by virtue of an overall displacement of the second driving mechanism on the frame.

14. The delivery apparatus for elongated medical instruments according to claim 12, wherein the second driving mechanism clamps one end of the second medical instrument, and the third driving mechanism is movably arranged on the frame, and clamps the other end of the second medical instrument and delivers the second medical instrument by virtue of displacement on the frame.

15. The delivery apparatus for elongated medical instruments according to claim 14, wherein an internal structure of the second driving mechanism comprises a second delivering assembly configured to deliver the second medical instrument and a second rotating assembly configured to rotate the first medical instrument.

16. The delivery apparatus for elongated medical instruments according to claim 15, wherein the second delivering assembly comprises a second roller set; wherein one end of the second medical instrument is delivered by virtue of a friction between the second roller set and the second medical instrument; and the third driving mechanism is driven to displace by a driving device arranged on the frame, and the other end of the second medical instrument is delivered by virtue of an overall displacement of the third driving mechanism on the frame.

17. The delivery apparatus for elongated medical instruments according to claim 15, wherein the second rotating assembly comprises a rotating unit, configured to drive a T valve to rotate, wherein the first medical instrument secured onto the T valve rotates under driving of the T valve.

18. The delivery apparatus for elongated medical instruments according to claim 1, wherein the first medical instrument is a first catheter, the second medical instrument is a second catheter, the third medical instrument is a first guidewire, and the fourth medical instrument is a second guidewire.

19. The delivery apparatus for elongated medical instruments according to claim 3, wherein the second driving mechanism and the third driving mechanism remain stationary relative to each other, and the third driving mechanism is attached to the second driving mechanism combined with the second driving mechanism 2 to form an integral structure.

20. The delivery apparatus for elongated medical instruments according to claim 3, wherein the second driving mechanism further comprises a rapid exchange delivering assembly, wherein the rapid exchange delivering assembly is configured to clamp a rapid exchange catheter.
